(19) United States
(12) Patent Application Publication
(10) Pub. No.: US 2007/0027211 A1
(43) Pub. Date: Feb. 1, 2007

(54) Process for production of bicalutamide

(75) Inventors: Zhi-Xian Wang, Brantford (CA); Yuan-Qiang Li, Brantford (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Apotex Pharmachem Inc.

(21) Appl. No.: 11/493,281

(22) Filed: Jul. 26, 2006

(30) Foreign Application Priority Data
Jul. 26, 2005 (CA) 2,513,356

(51) Int. Cl. A61K 31/277; C07C 253/30

(52) U.S. Cl. 514/521; 558/410

(57) ABSTRACT

A process which includes the reacting of sodium perborate with N-[4-cyano-3-(trifluoromethyl)phenyl]-3-[(4-fluorophenyl)thio]-2-hydroxy-2-methylpropanamide to form bicalutamide. The process is efficient, inexpensive, environmentally friendly and produces bicalutamide in good yield.

### FIELD OF THE INVENTION

The present invention relates to a new process for the synthesis of Bicalutamide.

### BACKGROUND OF THE INVENTION

N-[4-cyano-3-(trifluoromethyl)phenyl]-3-[(4-fluorophenyl)sulfonyl]-2-hydroxy-2-methyl-propanamide, is known as the compound Bicalutamide (I). It is commercially available as Casodex® which is a non-antiandrogen used in the treatment of prostate cancer.

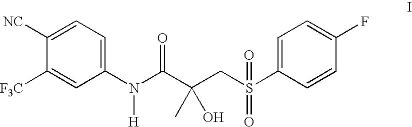

Various methods of synthesizing Bicalutamide are disclosed in U.S. Pat. No. 4,636,505, WO 01/00608 and U.S. Pat. No. 6,562,994. A common intermediate before the last step is N-[4-cyano-3-(trifluoromethyl)phenyl]-3-[(4-fluorophenyl)thio]-2-hydroxy-2-methylpropanamide, the thioether derivative of the formula (II), which is subsequently oxidized to produce Bicalutamide.

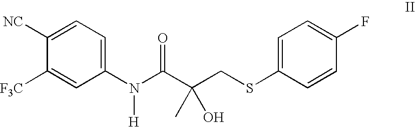

U.S. Pat. Nos. 4,636,505 and 6,562,994 describe a preferred process of preparing the precursor thioether (II) by reacting N-[4-cyano-3-(trifluoromethyl)phenyl]-2-methyloxiranecaboxamide of formula (III) with 4-fluorobenzenethiol in tetrahydrofuran in the presence of the very strong base, sodium hydride. Sodium hydride is a flammable solid, and is difficult to handle on large-scale as it can generate explosive hydrogen gas. When using tetrahydrofuran as the solvent, the work-up procedure is further complicated by the fact that the product II cannot be crystallized directly from the solution. Also tetrahydrofuran is an expensive solvent, which increases the cost for commercial-scale production.

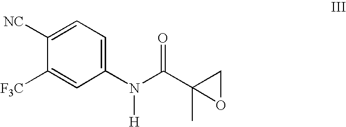

In addition, U.S. Pat. No. 6,562,994 also generally describes the use of other bases including alkali metal alkoxides, alkali metal amides and alkyllithiums, however sodium hydride is discussed as being more preferred. This disclosure also generally describes the use of only aprotic solvents, preferably ether based solvents such as the above mentioned tetrahydrofuran.

U.S. Pat. No. 4,636,505 describes that depending on the oxidizing agent and conditions used, a sulphinyl or a sulphonyl compound may be obtained when oxidizing the precursor N-[4-cyano-3-(trifluoromethyl)phenyl]-3-[(4-fluorophenyl)thio]-2-hydroxy-2-methylpropanamide (II) to obtain the final product. It describes a preferred process of oxidation wherein compound II is oxidized with m-chloroperbenzoic acid (m-CPBA) in methylene chloride to give the desired sulphonyl compound Bicalutamide. m-Chloroperbenzoic acid is a highly explosive and expensive reagent, and is, therefore, not a preferable reagent for use in commercial scale production. Furthermore, the use of halogenated organic solvents such as methylene chloride is harmful to the human body and the environment.

Patent No. WO 01/00608 discloses that Bicalutamide can be obtained preferably by oxidation of N-[4cyano-3-(trifluoromethyl)phenyl]-3-[(4-fluorophenyl)thio]-2-hydroxy-2-methyl-propanamide (II) with Oxone® (a combination of potassium hydrogenpersulfate/potassium hydrogensulfate/potassium sulfate) as the oxidizing agent. Due to the high molecular weight of Oxone®, a large amount of this reagent is necessary for the oxidation, and therefore a large amount of waste will also be produced. This also complicates the work-up procedure. For economic reasons, it is not advantageous to use Oxone® on a large scale.

U.S. Pat. No. 6,562,994 generally describes a process of oxidizing the thioether compound of formula II with a suitable oxidizing agent in the presence of aprotic solvents, preferably halogenated hydrocarbons. It teaches a preferred exemplified process of preparing Bicalutamide by oxidizing the thioether compound of formula II with a combination of hydrogen peroxide and trifluoroacetic anhydride in dichloromethane, which generates in situ trifluoroperacetic acid as an oxidant to give Bicalutamide in good yield. Though hydrogen peroxide is a low cost reagent, trifluoroacetic anhydride is an expensive chemical thereby increasing the cost of this route. In addition, this process suffers from the fact that trifluoroacetic anhydride is corrosive and hygroscopic, and cooling (−55° C.) is needed during the addition of trifluoroacetic anhydride to the mixture. Furthermore, the use of halogenated organic solvents such as methylene chloride is harmful to the human body and the environment. Overall this method is unsuitable for large-scale production.

U.S. Pat. No. 6,740,770 describes a method of producing Bicalutamide by oxidizing thioether (II) with hydrogen peroxide in the presence of sodium tungstate, phenylphosphonic acid and a phase transfer catalyst in ethyl acetate.

The addition of sodium tungstate, phenylphosphonic acid and a phase transfer catalyst increases the cost and complicates the work-up procedure. U.S. Pat. No. 6,740,770 also describes using mono-perphthalic acid as an oxidizing agent. Mono-perphthalic acid is not commercially available, and it is necessary to prepare it by mixing phthalic anhydride and hydrogen peroxide which results in an extra step and therefore increases the cost to the process.

Based on the disadvantages in the above processes, it would be highly desirable to have a simple, low cost, highly efficient and environmentally friendly process for the production of Bicalutamide thereby overcoming the deficiencies of the prior art.

### SUMMARY OF INVENTION

It is therefore one aspect of the invention to provide a novel process of producing N-[4-cyano-3-(trifluoromethyl)phenyl]-3-[(4-fluorophenyl)sulfonyl]-2-hydroxy-2-methyl-propanamide (Bicalutamide) of the formula of (I). The process provides a practical, efficient, economical, as well as being environmentally friendly production method as generally shown in the Scheme 1.

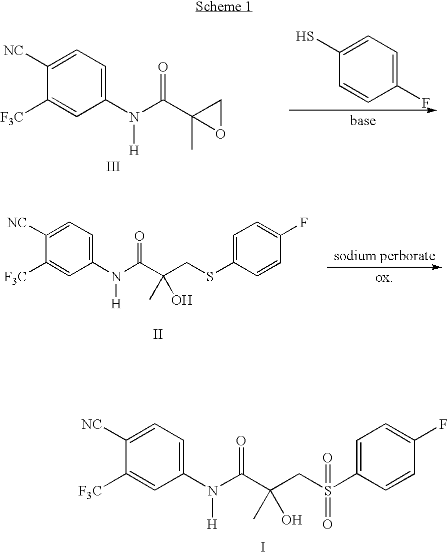

It is one aspect of the invention to provide for a process for the preparation of Bicalutamide which process comprises of reacting N-[4-cyano-3-(trifluoromethyl)phenyl]-2-methyloxiranecaboxamide with 4-fluorobenzenethiol in the presence of a base, water and a first solvent that is water miscible to form N-[4cyano-3-(trifluoromethyl)phenyl]-3[(4-fluorophenyl)thiol-2-hydroxy-2-methylpropanamide; and reacting the N-[4-cyano-3(trifluoromethyl)phenyl]-3-[(4-fluorophenyl)thio]-2-hydroxy-2-methylpropanamide with sodium perborate in a second solvent.

It is another aspect of the invention to provide for a process for the preparation of Bicalutamide which process comprises the oxidizing of N-[4cyano-3-(trifluoromethyl)phenyl]-3-[(4-fluorophenyl)thio]-2-hydroxy-2-methylpropanamide with sodium perborate in a solvent.

It is yet another aspect of the invention to provide for a process wherein the first solvent is selected from the group consisting of C1-C4 alkyl alcohol, an alkyl cyclic or acyclic amides, C3-C8 cyclic or acyclic sulfoxides and sulfones, alkyl nitriles; preferably methanol, ethanol, n-propanol, iso-propanol, n-butanol, N,N,-dimethylformamide, N,N-dimethylacetamide, 1-methyl-2-pyrrolidinone, dimethylsulfoxide, tetramethylene sulfone or acetonitrile.

It is yet another aspect of the invention to provide for a process wherein the base is selected from the group consisting of an alkali metal hydroxide; an alkali metal carbonate; or an alkali alkylate, preferably sodium hydroxide, potassium hydroxide, lithium hydroxide, sodium carbonate, potassium carbonate, lithium carbonate, sodium methoxide, sodium ethoxide, sodium tert-butoxide, potassium tert-butoxide or an aqueous solution of an alkali metal hydroxide such as sodium hydroxide or potassium hydroxide.

It is yet another aspect of the invention to provide a process wherein the second solvent or solvent in which the oxidization takes place is selected from the group consisting of C1-C4 carboxylic acid; alkyl cyclic or acyclic amides; alkyl cyclic and acyclic sulfoxide, preferably formic acid, acetic acid, propanoic acid, trifluoroacetic acid, N,N-dimethylformamide, N,N-dimethylacetamide, N-methyl-2-pyrrolidinone, dimethyl sulfoxide, and tetramethylene sulfone.

### DETAILED DESCRIPTION OF PREFERRED EMBODIMENTS

The present invention provides a novel process of producing N-[(cyano-3-(trifluoromethyl)phenyl]-3-[(4-fluorophenyl)sulfonyl]-2-hydroxy-2-methyl-propanamide (Bicalutamide) of the formula of (I). The process is industrially practical, efficient, safe and economical, as well as being environmentally friendly. The general method as shown in the Scheme 1.

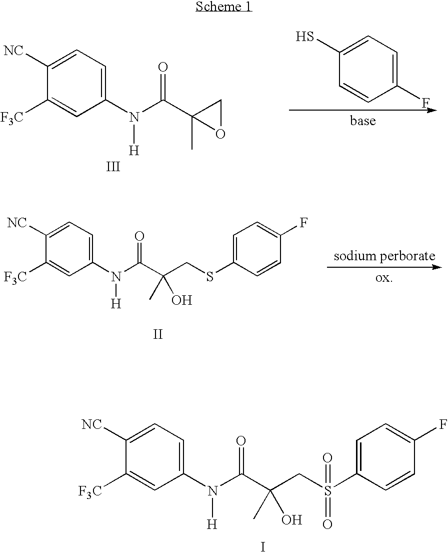

The thioether compound of formula II can be produced by combining the compound of the formula III with 4-fluorobenzenethiol in the presence of a suitable base in a suitable water miscible solvents together with water. The thioether compound of formula II is produced in high yield and purity.

The suitable water miscible solvents include both aprotic and protic solvents which include C1-C4 alkyl alcohols such as methanol, ethanol, n-propanol, iso-propanol, n-butanol; alkyl cyclic and acyclic amides such as N,N-dimethylformamide, N,N-dimethylacetamide and 1-methyl-2-pyrrolidinone; C3-C8 cyclic or acyclic alkyl sulfoxides and sulfones such as dimethylsulfoxide and tetramethylene sulfone; and alkyl nitrites such as acetonitrile. The most preferred solvents are the C1-C4 alkyl alcohols as the solvent, it simplifies the work-up procedure, and the compound of the formula II can be isolated by direct crystallization from the reaction solution, without the need for liquid-liquid extraction. Furthermore, C1-C4 alkyl alcohols are less expensive and easier to handle than the previously taught use of tetrahydrofuran and are preferable for large-scale production. The most preferable solvent is methanol. The amount of solvent preferably ranges from 0.5 volumes to 20 volumes relative to compound III, more preferably from 1 volume to 5 volumes.

The suitable bases need not be as strong as the previously used sodium hydride. They include alkali metal hydroxides, such as sodium hydroxide, potassium hydroxide, and lithium hydroxide; alkali metal carbonates such as sodium carbonate, potassium carbonate and lithium carbonate; and alkali alkylates such as sodium methoxide, sodium ethoxide, sodium tert-butoxide, potassium tert-butoxide and the like. The most preferred bases are alkali metal hydroxides such as sodium hydroxide and potassium hydroxide. Sodium hydroxide or aqueous sodium hydroxide solutions are the most preferred. The concentration of sodium hydroxide and potassium hydroxide preferably ranges from 5 weight percent to 50 weight percent, more preferably from 25 weight percent to 50 weight percent. The amount of base preferably ranges between 1.0 to 2.0 equivalents relative to compound III, more preferably between 1.0 to 1.2 equivalents.

The base reacts with 4-fluorobenzenethiol in the solvent to give a 4-fluorobenzenethiol alkali salt solution, which further reacts with compound III to give compound II. In order to form the 4-fluorobenzenethiol alkali salt solution, the aqueous base solution, 4-fluorobenzenethiophenol and solvent may be added in any order. The preferred procedure is that the aqueous base solution is added portionwise to a solution of 4-fluorobenzenethiol in the water-miscible solvent. The temperature of mixing the base with 4fluorobenzenethiol is preferably between −10° C. and 65° C., and more preferably between 0 and 20° C. After the 4-fluorobenzenethiol alkali salt forms, compound III may be added to the mixture as a solid or as a solution dissolved in the water miscible solvent. The reaction temperature is preferably between −10° C. and 65° C., and more preferably the temperature is between 0 and 25° C.

Although the compound of formula II can be separated from the reaction by liquid-liquid extraction, which is described in U.S. Pat. Nos. 6,562,994 and 6,740,770, where tetrahydrofuran is used as solvent, it is desirable in large-scale production to isolate the product directly from the reaction mixture through precipitation. The compound of the formula II can be directly precipitated from the reaction mixture by the addition of an anti-solvent. The preferred anti-solvent is water or C5-C12 hydrocarbons. The more preferred solvents are water, toluene, xylenes, heptanes, hexanes, and the like. The ratio of reaction solvent and anti-solvent is preferably between 3:1 and 1:100 (v/v), and more preferably between 1:1 and 1:20 (v/v). The compound of the formula II may be isolated by filtration in high yield and purity.

Unexpectedly, it was discovered that bicalutamide can be obtained in high purity and yield in an efficient process wherein the oxidation of the thioether compound of formula II is obtained using sodium perborate in a suitable solvent. Sodium perborate can be in its anhydrous, mono, di, tri and tetrahydrated forms. Sodium perborate is a very cheap, large-scale industrial chemical (over 500,000 tons per annum) and is exceptionally stable in its solid form without shock sensitivity. It is relatively non-toxic and used primarily as a source of “active oxygen” in detergents and as a mild antiseptic and a mouthwash. In addition to providing bicalutamide in high purity and yield, neither it nor the product of its reduction products is regarded as a hazardous chemical. As such, the by-products of sodium perborate reaction are innocuous, and hence there is no effluent problem in commercial-scale application. The amount of the oxidizing reagent relative to compound II is preferably between 2.0 and 10 equivalents, more preferably is between 2.2 and 3.0 equivalents.

The suitable solvents for this oxidation step include C1-C4 carboxylic acid such as formic acid, acetic acid, propanoic acid, trifluroacetic acid, or their mixtures with water; alkyl cyclic and acyclic amides such as N,N-dimethylformamide, N,N-dimethylacetamide and 1-methyl-2-pyrrolidinone, or their mixtures with water; cyclic or aclyclic alkyl sulfoxides such as dimethyl sulfoxide and tetramethylene sulfone, or their mixtures with water. The preferred solvents are acetic acid, formic acid, propanoic acid, and their mixture with water. The most preferred solvent is acetic acid and its mixture with water. The preferred ratio between acetic acid and water is between 1:0 and 1:10, more preferably the ratio is between 1:0 and 1:2. The amount of solvent ranges preferably between 0.5 volumes to 20 volumes relative to a volume of compound III, more preferably between 1 volume to 5 volumes. Preferably the oxidation reaction takes place between 0 and 120° C., more preferably between 25° C. and 100° C., an d most preferably between 70° C. and 90° C.

Although the Bicalutamide can be separated from the reaction by normal liquid-liquid extraction or column chromatography, it is desirable for commercial-scale production to isolate the product directly from the reaction mixture through precipitation. To this end, the compound of formula II can be directly precipitated from the reaction mixture by the addition of an anti-solvent. The preferred anti-solvents are water, C5-C12 alkyl or aryl hydrocarbons, and C3-C8 alkyl ketones. The more preferred anti-solvents are water, toluene, xylenes, heptanes, hexanes, methyl ethyl ketone, and methyl isobutyl ketone. The most preferred anti-solvent is water. The preferred ratio between reaction solvent and anti-solvent is between 2:1 and 1:100 (v/v), and more preferably between 1:1 and 1:20 (v/v). The precipitation can be performed by addition of the Bicalutamide solution into anti-solvent or the addition of anti-solvent into Bicalutamide solution at any rate desired. Bicalutamide is collected by filtration in high purity and yield.

The following non-limiting examples further illustrate the present invention for the preparation of Bicalutamide.

### EXAMPLES

### Example 1

### Preparation of N-[4-cyano-3-(trifluoromethyl)phenyl]-3-[(4-fluorophenyl)thio]-2-hydroxy-2-methylpropanamide

A solution of 4-fluorobenzenethiol (21g) in methanol (65 ml) was cooled to 0° C. and aqueous 50% sodium hydroxide (14 g) was added portionwise. The mixture was stirred at 0° C. for 30 minutes, then at 25° C. for 1 hour. To the mixture, N-[4-cyano-3-trifluoromethylphenyl]-2-methyloxiranecaboxamide (40 g) was added and the resulting mixture was stirred at room temperature for 2h. The reaction was determined to complete by TLC. Water (100 ml) was added to the mixture, followed by concentrated hydrochloric acid to a pH below 7. The solution was distilled under vacuum until no methanol distilled ceased, and the resulting suspension was stirred at 5° C. for 3 hours. The solid was collected by filtration and rinsed with water (2×40 ml). The solid was dried under vacuum at 50-60° C. to give 58 g (98%) of N-[4-cyano-3-(trifluoromethyl)phenyl]-3-[(4-fluorophenyl)thio]-2-hydroxy-2-methylpropanamide.

### Example 2

### Preparation of N-[4cyano-3-(trifluoromethyl)phenyl]-3-[(4-fluorophenyl)sulfonyl]-2-hydroxy-2-methyl-propanamide

A mixture of N-[4-cyano-3-(trifluoromethyl)phenyl]-3-[(4-fluorophenyl)thio]-2-hydroxy-2-methylpropanamide (50 g) and sodium perborate monohydrate (31 g) in acetic acid (200 ml) was heated to 80° C. for 3 hours. Reaction completion was determined by TLC. The mixture was cooled to 0° C., water (250 ml) was added, and the solid was collected by filtration. The crude product was recrystallized from ethyl acetate/heptanes to give 50 g (92%) of N-[4-cyano-3-(trifluoromethyl)phenyl]-3-(4-fluorophenyl)sulfonyl]-2-hydroxy-2-methyl-propanamide in 99.5% purity.

We claim:

1. A process for the preparation of Bicalutamide which process comprises of:
(a) reacting N-[4-cyano-3-(trifluoromethyl)phenyl]-2-methyloxiranecaboxamide with 4-fluorobenzenethiol in the presence of a base, water and a first solvent that is water miscible to form N-[4-cyano-3-(trifluoromethyl)phenyl]-3[(4-fluorophenyl)thio]-2-hydroxy-2-methylpropanamide; and
(b) reacting said N-14-cyano-3-(trifluoromethyl)phenyl]-3-[(4-fluorophenyl)thio]-2-hydroxy-2-methylpropanamide with sodium perborate in a second solvent.

2. The process according to claim 1 wherein said first solvent is selected from the group consisting of C1-C4 alkyl alcohol; an alkyl cyclic or acyclic amides; C3-C8 cyclic or acyclic sulfoxides and sulfones; and C2-C5 alkyl nitrites.

3. The process according to claim 1 wherein said first solvent is selected from the group consisting of methanol, ethanol, n-propanol, iso-propanol, and n-butanol.

4. The process of claim 1 wherein said first solvent is present in an amount between 1 to 5 volumes relative to said N-[4-cyano-3-(trifluoromethyl)phenyl]-2-methyloxiranecaboxamide.

5. The process according to claim 1 wherein said base is selected from the group consisting of an alkali metal hydroxide; an alkali metal carbonate; or alkali alkylate.

6. The process according to claim 5 wherein said base is present in a ratio between 1 to 2 equivalents relative to said N-[4-cyano-3-(trifluoromethyl)phenyl]-3-[(4-fluorophenyl)thio-2-hydroxy-2-methylpropanamide.

7. The process according to claim 6 wherein said ratio is between 1 to 1.2 equivalents.

8. The process according to claim 1, wherein said base is an aqueous solution of an alkali metal hydroxide selected from the group consisting of a sodium hydroxide and potassium hydroxide.

9. The process according to claim 8 wherein the concentration of said aqueous solution is between 5 and 50 wt. percent.

10. The process according to claim 7 wherein said concentration is between 25 and 50 wt. percent.

11. The process according to claim 1, wherein said second solvent is selected from the group consisting of C1-C4 carboxylic acid.

12. The process according to claim 11, wherein said second solvent comprises a solvent selected from the group consisting of formic acid, acetic acid, propanoic acid, and trifluoroacetic acid.

13. The process according to claim 11 wherein said second solvent also comprises water.

14. The process according to claim 11 wherein said second solvent also comprises water and the ratio of said water to said solvent is less than 2 to 1 parts by weight.

15. The process according to claim 1 wherein said sodium perborate is present in either its monohydrate, dihydrate, trihydrate or tetrahydrate forms.

16. A process for the preparation of Bicalutamide, said process comprising the oxidizing of N-[4-cyano-3(trifluoromethyl)phenyl]-3[(4-fluorophenyl)thio]-2-hydroxy-2-methylpropanamide with sodium perborate in a solvent.

17. The process according to claim 16, wherein said solvent is selected from the group consisting of C1-C4 carboxylic acid.

18. The process according to claim 17, wherein said solvent comprises a solvent selected from the group consisting of formic acid, acetic acid, propanoic acid, and trifluoroacetic acid.

19. The process according to claim 17 wherein said solvent also comprises water.

20. The process according to claim 19 wherein said solvent also comprises water and the ratio of said water to said solvent is less than 2 to 1 parts by weight.

21. The process according to claim 16 wherein said sodium perborate is present in either its monohydrate, dihydrate, trihydrate or tetrahydrate forms.
